(19) United States
(12) Patent Application Publication
(10) Pub. No.: US 2007/0083121 A1
(43) Pub. Date: Apr. 12, 2007

(54) TRANSESOPHAGEAL ULTRASOUND PROBE WITH REDUCED WIDTH

(76) Inventors: Harold M. Hastings, Garden City, NY (US); Scott L. Roth, East Hills, NY (US)

Correspondence Address: PROSKAUER ROSE LLP;PATENT DEPARTMENT, 1585 BROADWAY, NEW YORK, NY, 10036-8299, US

(21) Appl. No.: 11/534,403

(22) Filed: Sep. 22, 2006

Related U.S. Application Data
(60) Provisional application No. 60/721,032, filed on Sep. 26, 2005.

(51) Int. Cl. A61B 8/14

(52) U.S. Cl. 600/459

(57) ABSTRACT

The azimuthal aperture of the transducer in a transesophageal echocardiography probe can be maximized, for a given probe diameter, by eliminating unnecessary structures in the azimuthal direction.

### CROSS REFERENCE TO RELATED APPLICATIONS

This application claims the benefit of US provisional application No. 60/721,032, filed Sep. 26, 2005.

### BACKGROUND

In order to obtain repeatedly usable images from conventional transesophageal echocardiography (TEE) transducers, the azimuthal aperture of the transducers must be quite large (e.g., 10-15 mm in diameter for adults), which requires a correspondingly large probe. Because of this large probe, conventional TEE often requires anesthesia, can significantly threaten the airway, and is not well suited for long-term monitoring of the heart.

### SUMMARY OF THE INVENTION

The outside width of the housing that contains the TEE transducer can be reduced by a small but nevertheless significant amount by eliminating unnecessary structures.

### BRIEF DESCRIPTION OF THE DRAWINGS

FIG. 1 is an overall block diagram of a system for monitoring cardiac function by direct visualization of the heart.

FIG. 2 is a more detailed view of the probe shown in the FIG. 1 embodiment.

FIG. 3 is a schematic representation of a displayed image of the trans-gastric short axis view (TGSAV) of the left ventricle.

FIG. 4 depicts the positioning of the transducer, with respect to the heart, to obtain the TGSAV.

FIG. 5 shows a plane that slices through the trans-gastric short axis of the heart.

FIGS. 6A, 6B, and 6C show a first preferred transducer configuration.

FIGS. 7A and 7B show a second preferred transducer configuration.

FIGS. 8A and 8B show one way to mount the transducer within the housing.

FIGS. 9A and 9B show another way to mount the transducer within the housing.

### DESCRIPTION OF THE PREFERRED EMBODIMENTS

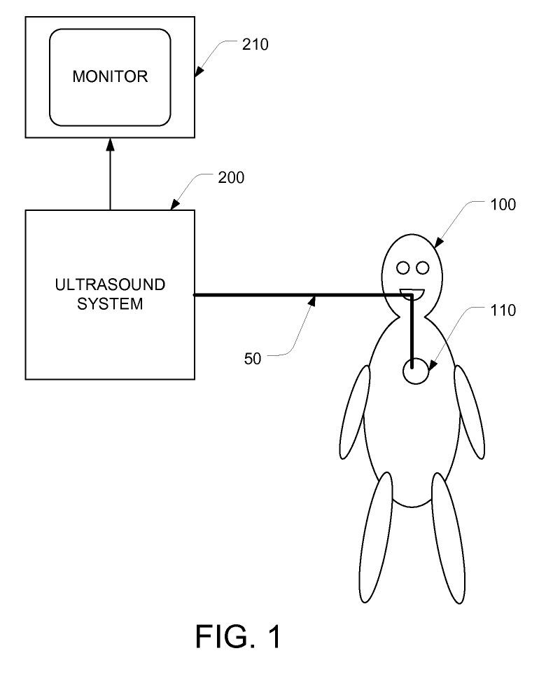

FIG. 1 is an overall block diagram of a system that may be used for continuous long term monitoring of cardiac function by direct visualization of the heart. An ultrasound system 200 is used to monitor the heart 110 of the patient 100 by sending driving signals into a probe 50 and processing the return signals received from the probe into images. The images generated by those algorithms are then displayed on a monitor 210, in any conventional manner. A number of techniques that enable a usable image to be obtained from a transducer with a small azimuthal aperture are described in U.S. patent application Ser. No. 10/997,059, filed Nov. 24, 2004, which is incorporated herein by reference.

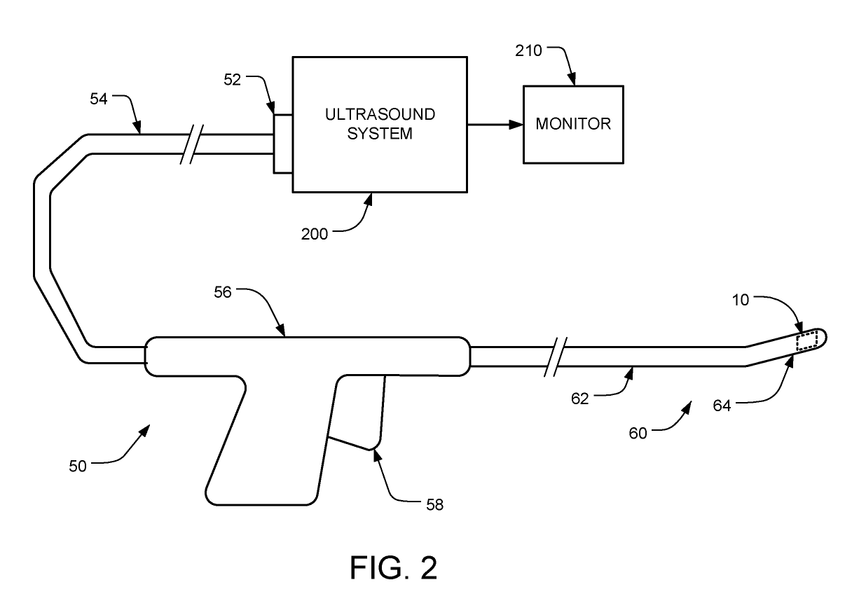

FIG. 2 shows more details of the probe 50, which is connected to the ultrasound system 200. At the distal end of the probe 50 there is a housing 60, and the ultrasound transducer 10 is located in the distal end 64 of the housing 60. The next portion is the flexible shaft 62, which is positioned between the distal end 64 and the handle 56. This shaft 62 should be flexible enough so that the distal end 64 can be positioned past the relevant anatomical structures to the desired location, and the handle 56 facilitates the positioning of the distal end 64 by the operator. Optionally, the handle 56 may contain a triggering mechanism 58 which the operator uses to bend the end of the housing 60 to a desired anatomical position as described below.

At the other end of the handle 56 is a cable 54, which terminates, at the proximal end of the probe 50, at connector 52. This connector 52 is used to connect the probe 50 to the ultrasound system 200 so that the ultrasound system 200 can operate the probe. Signals for the ultrasound system 200 that drive the transducer 10 travel through the probe 50 via appropriate wiring and any intermediate circuitry (not shown) to drive the transducer 10, and return signals from the transducer 10 similarly travel back through the probe 50 to the ultrasound system 200 where they are ultimately processed into images. The images are then displayed on the monitor 210 in a manner well known to persons skilled in the relevant art.

In the preferred embodiments, the housing 60 has an outer diameter of less than or equal to 7.5 mm. The probe contains the ultrasound transducer 10 and connecting wires, and the housing 60 can be passed through the mouth or nose into the esophagus and stomach.

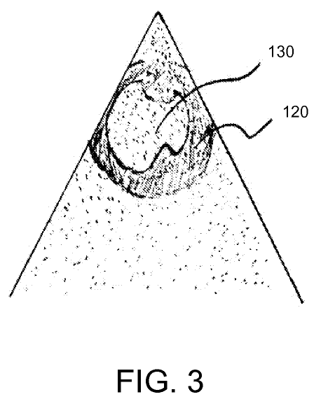

The returned ultrasound signals are processed in the ultrasound system 200 to generate an image of the heart. Preferably, additional signal processing is used to significantly improve image production, as described below. FIG. 3 shows a displayed image of the trans-gastric short axis view (TGSAV) of the left ventricle (LV), which is a preferred view that can be imaged using the preferred embodiments. The illustrated image of the TGSAV appears in a sector format, and it includes the myocardium 120 of the LV which surrounds a region of blood 130 within the LV. The image may be viewed in real time or recorded for later review, analysis, and comparison. Optionally, quantitative analyses of cardiac function may be implemented, including but not limited to chamber and vessel dimensions and volumes, chamber function, blood flow, filling, valvular structure and function, and pericardial pathology.

Unlike conventional TEE systems, the relatively narrow housing used in the preferred embodiments makes it possible to leave the probe in position in the patient for prolonged periods of time.

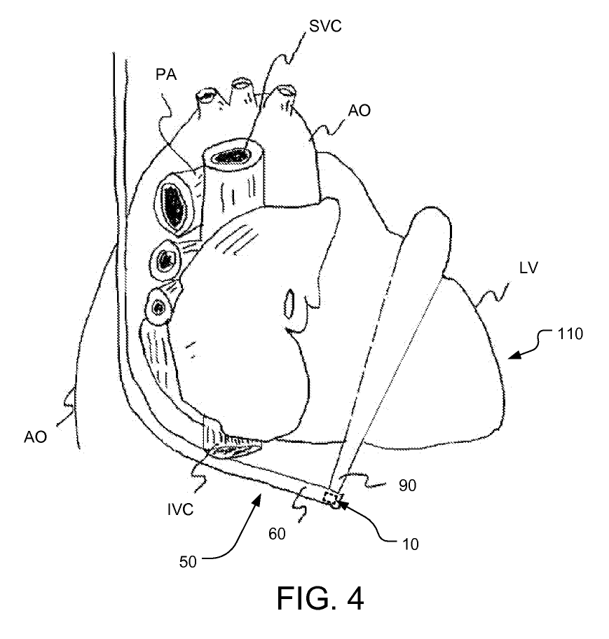

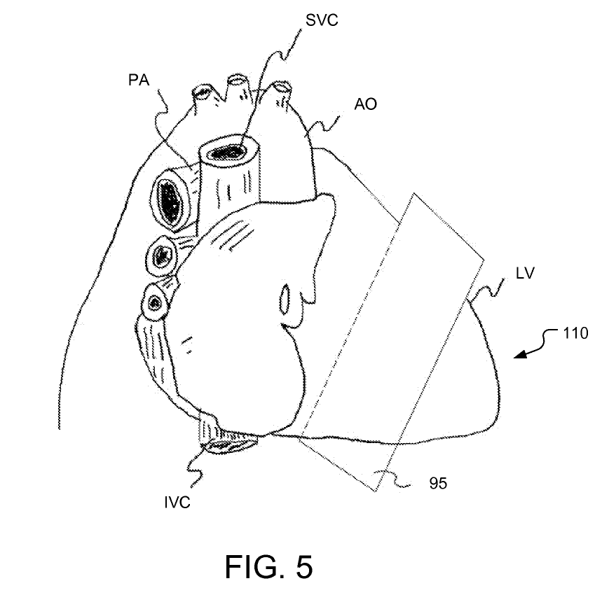

As best seen in FIGS. 4 and 5, the probe 50 is used to introduce and position the transducer 10 into a desired location within the patient's body. The orientation of the heart within the chest cavity is such that the apex of the left ventricle is positioned downward and to the left. This orientation results in the inferior (bottom) wall of the left ventricle being positioned just above the left hemidiaphragm, which is just above the fundus of the stomach. During operation, the transducer 10 emits a fan-shaped beam 90. Thus, positioning the transducer 10 in the fundus of the stomach with the fan-shaped beam 90 aimed through the left ventricle up at the heart can provide a trans-gastric short axis view image of the heart 110. The plane of the fan-shaped beam 90 defines the image plane 95 shown in FIG. 5. That view is particularly useful for monitoring the operation of the heart because it enables medical personnel to directly visualize the left ventricle, the main pumping chamber of the heart. Note that in FIGS. 4 and 5, AO represents the Aorta, IVC represents the Inferior Vena Cava, SVC represents the Superior Vena Cava, PA represents the pulmonary artery, and LV represents the left ventricle.

Other transducer positions may also be used to obtain different views of the heart, typically ranging from the mid-esophagus down to the stomach, allowing the operator to directly visualize most of the relevant cardiac anatomy. For example, the transducer 10 may be positioned in the lower esophagus, so as to obtain the conventional four chamber view. Transducer positioning in the esophagus would typically be done without fully flexing the probe tip, prior to advancing further into the stomach. Within the esophagus, desired views of the heart may be obtained by having the operator use a combination of some or all of the following motions with respect to the probe: advance, withdraw, rotate and slight flex.

For use in adults, the outer diameter of the housing 60 is preferably less than or equal to 7.5 mm, more preferably less than or equal to 6 mm, and is most preferably about 5 mm. This is significantly smaller than conventional TEE probes. This size reduction may reduce or eliminate the need for anesthesia, and may help expand the use of TEE for cardiac monitoring beyond its previous specialized, short-term settings. When a 5 mm housing is used, the housing is narrow enough to pass through the nose of the patient, which advantageously eliminates the danger that the patient will accidentally bite through the probe. Alternatively, it may be passed through the mouth like conventional TEE probes. Note that the 5 mm diameter of the housing is similar, for example, to typical NG (naso-gastric) tubes that are currently successfully used long-term without anesthesia in the same anatomical location. It should therefore be possible to leave the probe in place for an hour, two hours, or even six hours or more.

The housing wall is preferably made of the same materials that are used for conventional TEE probe walls, and can therefore withstand gastric secretions. The wiring in the probe that connects the transducer to the rest of the system may be similar to that of conventional TEE probes (adjusted, of course, for the number of elements). The housing is preferably steerable so that it can be inserted in a relatively straight position, and subsequently bent into the proper position after it enters the stomach. The probe tip may be deflected by various mechanisms including but not limited to steering or pull wires. In alternative embodiments, the probe may use an intrinsic deflecting mechanism such as a preformed element including but not limited to pre-shaped materials. Optionally, the probe (including the transducer housed therein) may be disposable.

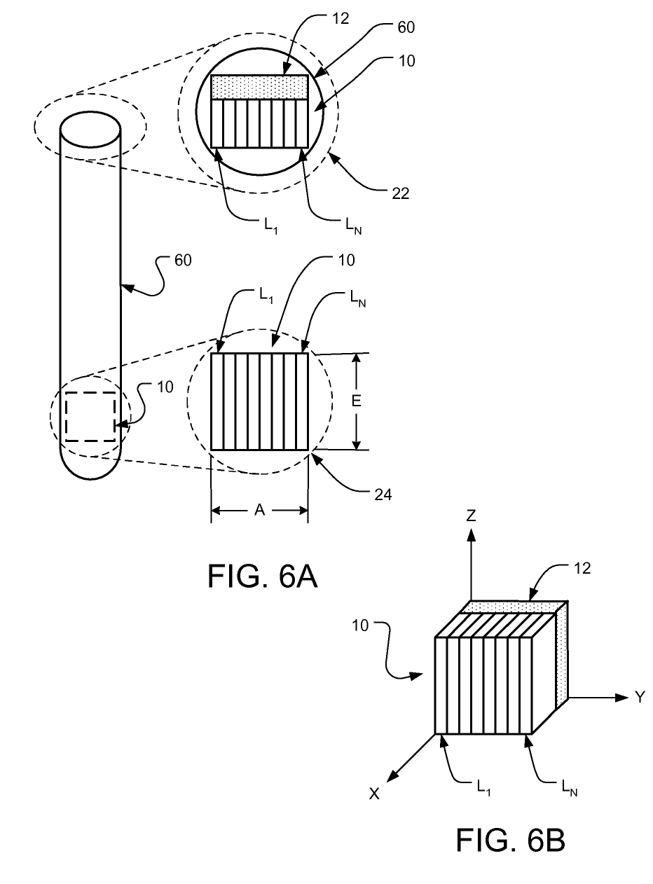

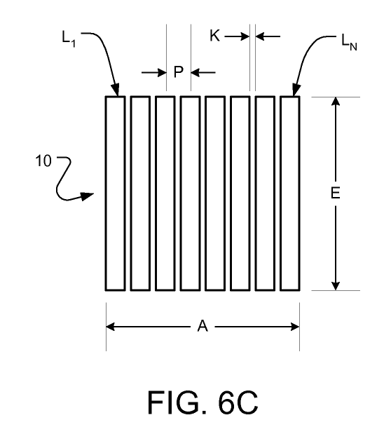

FIGS. 6A-6C depict a first preferred transducer 10. FIG. 6A shows the location of the transducer 10 in the distal end of the housing 60, and also includes a top view 22 of the transducer 10 surrounded by the wall of the housing 60 and a front cutaway view 24 of the transducer 10.

As best seen in FIG. 6B, the azimuth axis (Y axis) is horizontal, the elevation axis (Z axis) is vertical, and the X axis projects out of the page towards the reader. When steered straight forward by energizing the appropriate elements in the transducer, the beam will go straight out along the X axis. The steering signals can also send the beam out at angles with respect to the X axis, in a manner well know to persons skilled in the relevant arts.

The transducer 10 is preferably a phased array transducer made of a stack of N piezo elements L₁ . . . L_{N}, an acoustic backing 12, and a matching layer in the front (not shown), in a manner well known to those skilled in the relevant art. As understood by persons skilled in the relevant arts, the elements of phased array transducers can preferably be driven individually and independently, without generating excessive vibration in nearby elements due to acoustic or electrical coupling. In addition, the performance of each element is preferably as uniform as possible to help form a more homogeneous beam.

The preferred transducers use the same basic operating principles as conventional TEE transducers to transmit a beam of acoustic energy into the patient and to receive the return signal. However, while the first preferred transducer 10 shown in FIGS. 6A-6C shares many characteristics with conventional TEE transducers, the first preferred transducer 10 differs from conventional transducers in the following ways:

TABLE 1

| | conventional TEE | first preferred |
|---|---|---|
| Feature | transducer | transducer |
| Size in the transverse | 10-15 mm | about 4-5 mm |
| (azimuthal) direction | | |
| Number of elements | 64 | about 32-40 |
| Size in the elevation direction | 2 mm | about 4-5 mm |
| Front face aspect ratio | about 1:5 | about 1:1 |
| (elevation:transverse) | | |
| Operating frequency | 5 MHz | about 6-7.2 MHz |

In FIG. 6A, the elevation is labeled E and the transverse aperture is labeled A on the front cutaway view 24 of the transducer 10. The location of the wall of the housing 60 with respect to the transducer 10 can be seen in the top view 22.

FIG. 6C shows more details of the first preferred transducer 10. Note that although only eight elements are shown in all the figures, the preferred transducer actually has between about 32-40 elements, spaced at a pitch P on the order of 130 μm. Two particularly preferred pitches are approximately 125 μm (which is convenient for manufacturing purposes) and approximately 128 μm (0.6 wavelength at 7.2 MHz). When 32-40 elements are spaced at a 125 μm pitch, the resulting azimuth aperture A (sometimes simply called the aperture) of the transducer 10 will be between 4 and 5 mm. The reduced element count advantageously reduces the wire count (compared to conventional TEE transducers), which makes it easier to fit all the required wires into the narrower housing. The kerf K (i.e., the spacing between the elements) is preferably as small as practical (e.g., about 25-30 μm or less). Alternative preferred transducers may have between about 24-48 elements, spaced at a pitch between about 100-150 μm.

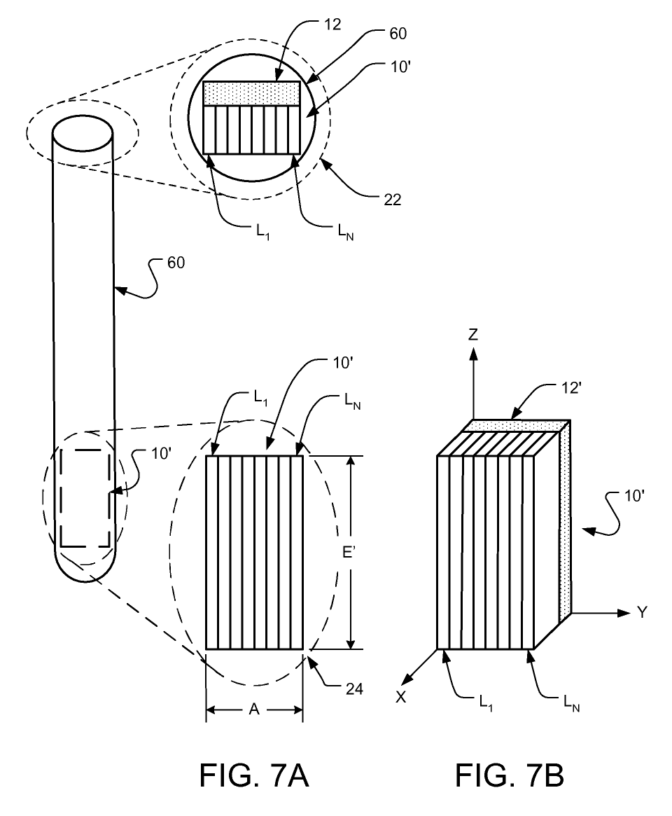

A second preferred transducer 10′ is shown in FIGS. 7A-7B. This transducer 10′ is similar to the first preferred transducer 10 described above in connection with FIGS. 6A-6C, except it is taller in the elevation direction. Similar reference numbers are used in both sets of figures to refer to corresponding features for both transducers. Numerically, the second transducer differs from conventional transducers in the following ways:

TABLE 2

| | conventional TEE | second preferred |
|---|---|---|
| Feature | transducer | transducer |
| Size in the transverse | 10-15 mm | about 4-5 mm |
| (azimuthal) direction | | |
| Number of elements | 64 | about 32-40 |
| Size in the elevation direction | 2 mm | about 8-10 mm |
| Front face aspect ratio | about 1:5 | about 2:1 |
| (elevation:transverse) | | |
| Operating frequency | 5 MHz | about 6-7.2 MHz |

In alternative embodiments, the transducer 10 may be built with a size in the elevation direction that lies between the first and second preferred transducers. For example, it may have a size in the elevation direction of about 7.5 mm, and a corresponding elevation:transverse aspect ratio of about 1.5:1.

The transducer 10 preferably has the same transverse orientation (with respect to the axis of the housing 60) as conventional TEE transducers. When the transducer is positioned in the stomach (as shown in FIG. 4), the image plane (azimuthal/radial plane) generated by the transducer intersects the heart in the conventional short axis cross-section), providing the trans-gastric short axis view of the heart, as shown in FIGS. 3 and 5. The transducer is preferably as wide as possible in the transverse direction within the confines of the housing. Referring now to the top view 22 in FIG. 6A, two examples of transducers that will fit within a 5 mm housing are provided in the following table, along with a third example that fits in a housing that is slightly larger than 5 mm:

TABLE 3

| | first | second | third |
|---|---|---|---|
| Parameter | example | example | example |
| number of elements in the transducer | 38 | 36 | 40 |
| a (azimuthal aperture) | 4.75 mm | 4.50 mm | 5.00 mm |
| b (thickness) | 1.25 mm | 2.00 mm | 2.00 mm |
| c (inner diameter of housing at the | 4.91 mm | 4.92 mm | 5.39 mm |
| transducer) | | | |
| housing wall thickness | 0.04 mm | 0.04 mm | 0.04 mm |
| outer diameter of housing | 4.99 mm | 5.00 mm | 5.47 mm |

Referring now to the top view 22 in FIG. 7A, the three examples in Table 3 are also applicable for fitting the second preferred transducer 10′ within a 5-5.5 mm housing.

The above-describe embodiments assume that the housing is round. However, other shaped housings may also be used to house the transducer, including but not limited to ellipses, ovals, etc. In such cases, references to the diameter of the housing, as used herein, would refer to the diameter of the smallest circle that can circumscribe the housing. To account for such variations in shape, the housing may be specified by its outer perimeter. For example, a 5 mm round housing would have a perimeter of 5 p mm (i.e., about 16 mm). When a rectangular transducer is involved, using an oval or elliptical housing can reduce the outer perimeter of the housing as compared to a round housing. For example, an oval that is bounded by a 6 mm×2 mm rectangle with its corners rounded to a radius of 0.5 mm contains a 5 mm×2 mm rectangular region, which can hold the third example transducer in Table 3. Allowing for a 0.04 mm housing wall thickness yields an outer perimeter of 15.4 mm, which is the same outer perimeter as a 4.9 mm diameter circle. The following table gives the outer perimeters that correspond to some of the diameters discussed herein:

TABLE 4

| outer diameter | outer perimeter |
|---|---|
| 2.5 mm | 8 mm |
| 4 | 13 |
| 5 | 16 |
| 6 | 19 |
| 7.5 | 24 |

Since the characteristics of the last one or two elements at each end of the transducer may differ from the characteristics of the remaining elements (due to differences in their surroundings), the last two elements on each side may be “dummy” elements. In such a case, the number of active elements that are driven and used to receive would be the total number of element (shown in Table 3) minus four. Optionally, the wires to these dummy elements may be omitted, since no signals need to travel to or from the dummy elements. Alternatively, the wires to may be included and the last two elements may be driven, with the receive gain for those elements severely apodized to compensate in part for their position at the ends of the transducer.

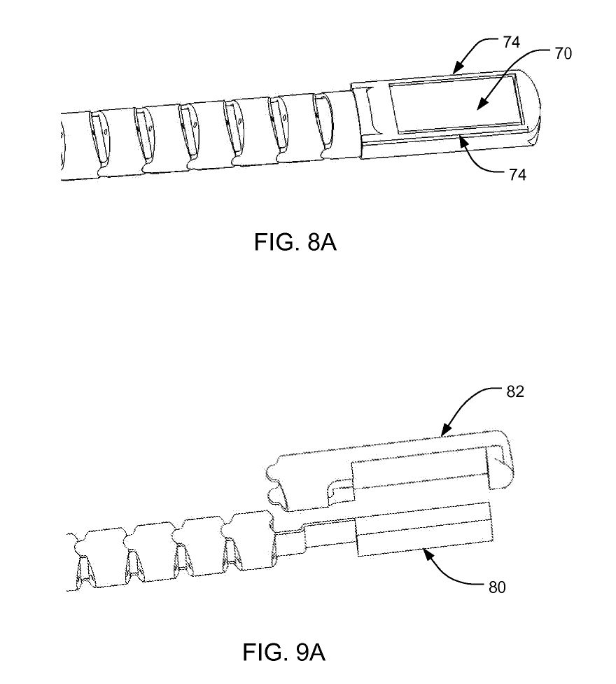

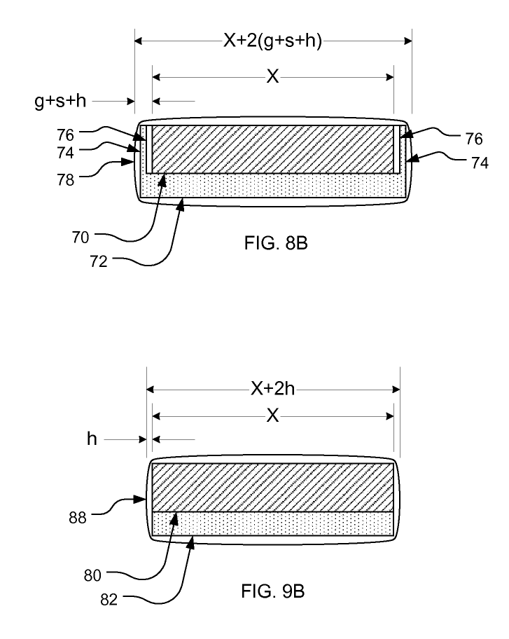

The ultrasound TEE transducers described herein may be mounted in a well as shown in FIGS. 8A and 8B, so that the transducer 70 sits on the bottom of the well 72, between the sidewalls 74. However, when they are so mounted, the sidewalls 74 of the well add to the width of the housing in the azimuthal direction. This is best seen in FIG. 8B, which is a cross section of the probe passing through the center of the transducer, with the azimuthal axis running horizontally and the elevation axis running perpendicular to the page. For this embodiment, the total width of the housing in the azimuthal direction can be computed using the formula W_{TOTAL}=X+2×(g+s+h), where X is the width of the transducer 70 in the azimuthal direction; s is the width of the sidewalls 74 of the well; g is the width of the gap 76 between the side of the transducer 70 and the sidewalls 74; and h is the width of the housing walls 78. The housing is not pictured in FIG. 8A, but a suitable housing is needed to protect the internal components, as will be understood by persons skilled in the relevant arts. Note that in this embodiment, it will not be possible to achieve the values described in table 3 above.

In an alternative embodiment, the total width of the housing in the azimuthal direction is reduced as compared to the FIG. 8 embodiment by mounting the transducer 80 on the surface of a paddle 82 that has no sidewalls (e.g., using a preferably very thin layer of a suitable adhesive). FIG. 9A is an exploded view of this configuration, and FIG. 9B is a cross section of a probe passing through the center of the transducer, with the azimuthal axis running horizontally and the elevation axis running perpendicular to the page. For this embodiment, the total width of the housing in the azimuthal direction can be computed using the formula W_{TOTAL}=X+2 h, where X is the width of the transducer 80 in the azimuthal direction; and h is the width of the housing walls 88. h is preferably less than or equal to 0.1 mm, and more preferably less than or equal to 0.05 mm. Thus, the housing in this embodiment is thinner than the housing depicted in FIGS. 8A and 8B by 2×(g+s). In this embodiment, it should be possible to achieve the values described in table 3 above.

This added reduction in the azimuthal direction is obtained without adversely impacting the resolution or depth of penetration that can be achieved using the probe (since the width of the transducer itself remains unchanged). This reduced width housing can help further improve ease of insertion, minimize airway restriction, optimize patient comfort, and minimize the need for anesthesia or sedation. Moreover, eliminating the sidewalls in this embodiment can advantageously improve heat conduction from the acoustic block (which generates heat) through the walls of the housing, thereby reducing the face temperature (typically the highest temperature on the outside of the housing) for a given operating power, or allowing higher power for a given face temperature.

If desired, the preferred embodiments described above may be scaled down for neonatal or pediatric use. In such cases, a transducer that is between about 2.5 and 4 mm in the azimuthal direction is preferable, with the elevation dimension scaled down proportionally. Because less depth of penetration is required for neonatal and pediatric patients, the operating frequency may be increased. This makes λ smaller, which permits the use of a smaller transducer element spacing (pitch), and a correspondingly larger number of elements per mm in the transducer. When such a transducer is combined with the above-described techniques, the performance should meet or surpass the performance of conventional 7.5 mm TEE probes for neonatal and pediatric uses.

The embodiments described herein may also be used in non-cardiac applications. For example, the probe could be inserted into the esophagus to monitor the esophagus itself, lymph nodes, lungs, the aorta, or other anatomy of the patient. Alternatively, the probe could be inserted into another orifice (or even an incision) to monitor other portions of a patient's anatomy.

Numerous other modifications to the above-described embodiments will be apparent to those skilled in the art, and are also included within the purview of the invention.

We claim:

1. An ultrasound probe for use with an ultrasound system comprising
a housing having a flexible shaft, a distal end, and a housing wall; and
an ultrasound transducer housed within the distal end of the housing, the transducer having a proximal end, a distal end, a front face from which an ultrasound beam emanates, a rear face opposite to the front face, and lateral sides, wherein the transducer is mounted within the housing so that the lateral sides of the transducer are in direct contact with the housing wall.

2. The ultrasound probe of claim 1, wherein the rear face of the transducer is supported by a paddle-shaped member.

3. The ultrasound probe of claim 1, wherein the rear face of the transducer is mounted on a paddle-shaped member using an adhesive.

4. The ultrasound probe of claim 1, wherein the transducer is a phased-array transducer that is transversely oriented with respect to a proximal-distal axis of the housing.

5. The ultrasound probe of claim 1, wherein the shaft has an outer diameter of less than or equal to 7.5 mm and the distal end has an outer diameter of less than or equal to 7.5 mm.

6. The ultrasound probe of claim 1, wherein the shaft has an outer diameter of less than or equal to 6 mm and the distal end has an outer diameter of less than or equal to 6 mm.

7. The ultrasound probe of claim 1, wherein the distal end has an outer diameter of about 5 mm.

8. The ultrasound probe of claim 1, wherein the transducer is a phased-array transducer that is transversely oriented with respect to a proximal-distal axis of the housing, wherein the rear face of the transducer is supported by a paddle-shaped member, wherein a ratio of the size of the transducer in the proximal-distal direction to the size of the transducer in the transverse direction is at least 1:1, and wherein the shaft has an outer diameter of less than or equal to 6 mm and the distal end has an outer diameter of less than or equal to 6 mm.

9. An ultrasound probe for use with an ultrasound system comprising
a housing having a flexible shaft, a distal end, and a housing wall; and
an ultrasound transducer housed within the distal end of the housing, the transducer having a proximal end, a distal end, a front face from which an ultrasound beam emanates, a rear face opposite to the front face, and lateral sides, wherein the transducer is mounted within the housing with no rigid structures disposed between the lateral sides of the transducer and the housing wall.

10. The ultrasound probe of claim 9, wherein the transducer is mounted within the housing with no gaps disposed between the lateral sides of the transducer and the housing wall.

11. The ultrasound probe of claim 10, wherein the rear face of the transducer is supported by a paddle-shaped member.

12. The ultrasound probe of claim 10, wherein the rear face of the transducer is mounted on a paddle-shaped member using an adhesive.

13. The ultrasound probe of claim 10, wherein the transducer is a phased-array transducer that is transversely oriented with respect to a proximal-distal axis of the housing.

14. The ultrasound probe of claim 10, wherein the transducer is a phased-array transducer that is transversely oriented with respect to a proximal-distal axis of the housing, wherein the rear face of the transducer is supported by a paddle-shaped member, wherein a ratio of the size of the transducer in the proximal-distal direction to the size of the transducer in the transverse direction is at least 1:1, and wherein the shaft has an outer diameter of less than or equal to 6 mm and the distal end has an outer diameter of less than or equal to 6 mm.

15. An ultrasound probe for use with an ultrasound system comprising
a housing having a flexible shaft, a distal end, and a housing wall, the housing wall having an outer surface; and
an ultrasound transducer housed within the distal end of the housing, the transducer having a proximal end, a distal end, a front face from which an ultrasound beam emanates, a rear face opposite to the front face, and lateral sides, wherein the transducer is mounted within the housing with the lateral sides of the transducer positioned less than or equal to 0.1 mm away from the outer surface of the housing wall.

16. The ultrasound probe of claim 15, wherein the transducer is mounted within the housing with the lateral sides of the transducer positioned less than or equal to 0.05 mm away from the outer surface of the housing wall.

17. The ultrasound probe of claim 15, wherein the rear face of the transducer is supported by a paddle-shaped member.

18. The ultrasound probe of claim 15, wherein the rear face of the transducer is mounted on a paddle-shaped member using an adhesive.

19. The ultrasound probe of claim 15, wherein the transducer is a phased-array transducer that is transversely oriented with respect to a proximal-distal axis of the housing.

20. The ultrasound probe of claim 15, wherein the transducer is a phased-array transducer that is transversely oriented with respect to a proximal-distal axis of the housing, wherein the rear face of the transducer is supported by a paddle-shaped member, wherein a ratio of the size of the transducer in the proximal-distal direction to the size of the transducer in the transverse direction is at least 1:1, and wherein the shaft has an outer diameter of less than or equal to 6 mm and the distal end has an outer diameter of less than or equal to 6 mm.

21. The ultrasound probe of claim 20, wherein the transducer is mounted within the housing with the lateral sides of the transducer positioned less than or equal to 0.05 mm away from the outer surface of the housing wall.
